(19) United States
(12) Patent Application Publication
(10) Pub. No.: US 2015/0037257 A1
(43) Pub. Date: Feb. 5, 2015

(54) COMPOSITIONS AND METHODS FOR HIGH-THROUGHPUT SCREENING IN SKIN FIBROBLASTS WITH AN ALPHA-SYNUCLEIN TRIPLICATION

(71) Applicants: Sally Mak, Sunnyvale, CA (US); Birgitt Schüle, Sunnyvale, CA (US); J. William Langston, Sunnyvale, CA (US)

(72) Inventors: Sally Mak, Sunnyvale, CA (US); Birgitt Schüle, Sunnyvale, CA (US); J. William Langston, Sunnyvale, CA (US)

(21) Appl. No.: 14/125,909

(22) PCT Filed: Jun. 15, 2012
(86) PCT No.: PCT/US12/42815

§ 371 Date: Aug. 25, 2014

Related U.S. Application Data
(60) Provisional application No. 61/497,617, filed on Jun. 16, 2011.

(51) Int. Cl. G01N 33/50; C12Q 1/68; C12N 5/077

(52) U.S. Cl. CPC G01N 33/5023; G01N 33/5044; C12N 5/0656; C12Q 1/6883; C12Q 2600/158; C12Q 2600/136; G01N 2800/2835; G01N 2333/904; G01N 2500/10
USPC 424/9.2; 424/133.1; 424/130.1; 514/44 A; 514/44 R; 514/17.7; 435/29; 435/6.13; 435/7.1; 435/325; 435/366; 506/10; 506/9; 435/6.12; 435/6.11; 506/2; 435/26

(57) ABSTRACT

Human-derived fibroblast cells with copy number variation for alpha-synuclein, and methods of use thereof, are provided. For example, compositions and methods for high through-put screening of potential therapies for neurodegenerative disease such as Parkinson's disease are provided.

This application claims priority from U.S. Provisional Application 61/497,617, filed Jun. 16, 2011, the entire contents of each of which are hereby incorporated by reference in their entirety.

### BACKGROUND OF THE INVENTION

Parkinson's disease (PD) is a progressive neurodegenerative disease affecting 1-2% of the population over 65 years of age. It is estimated that the number of prevalent cases of PD world-wide will double by the year 2030. The increasing disability caused by the progression of disease burdens the patients, their caregivers, and society. Classic neuronal pathological features of PD include the loss of dopaminergic (DA) neurons in the substantia nigra (SN) and the presence of cytoplasmic inclusions known as Lewy bodies. Classical clinical features of PD include resting tremor, bradykinesia and rigidity, but the disease is now know to have wide variety of non-motor features such as autonomic dysfunction and dementia. Although the pattern of neuronal loss in PD is well characterized, the molecular mechanisms that lead to that cell death are still unknown. The majority of PD patients suffer from idiopathic disease with no clear etiology, and approximately 5% of patients present with familial PD.

Currently, there is no cure, early detection mechanism, preventative treatment, or effective way to slow disease progression. A model that replicates the fundamental features of the disease at the cellular level is needed. Thus, there remains a need for improved screening methods for compositions and methods useful in the cure, detection, preventative treatment, paliative treatment, or treatment to slow disease progression for Parkinson's disease.

### SUMMARY OF THE INVENTION

A method is disclosed for measuring efficacy of an agent in the treatment of neurodegenerative disease, the method comprising contacting the agent with fibroblast cells containing a copy number variation for alpha-synuclein; detecting a response in the cells; and comparing the response to control cells. In various embodiments, the neurodegenerative disease is Parkinson's disease or Parkinson's-related disease. For example, in various embodiments, the copy number variation is a deletion, an insertion, a complex multi-state variant, a substitution, a transition, a transversion, or a duplication, of one or more nucleotides in the gene for alpha-synuclein. Preferably, the copy number variation is alpha-synuclein triplication.

Also disclosed is a method of pre-clinical or clinical development of a therapeutic for neurodegenerative disease comprising measuring efficacy of more than one agent according to the methods described herein, selecting at least one agent based on the results, and administering at least one agent to an animal model of the neurodegenerative disease. In various embodiments, a method of high-throughput drug screening is disclosed comprising performing the methods described herein, wherein the efficacy of more than one agent is measured, such as a library of agents including a library of known compounds.

Also described herein is an agent for treatment of a neurodegenerative disease formulated in a composition comprising the agent and a carrier suitable for treatment of the neurodegenerative disease, wherein the agent has efficacy in the treatment of the neurodegenerative disease, wherein the efficacy is measured according to the methods described herein.

Also described herein is a culture of fibroblast cells comprising cell culture media and further comprising fibroblast cells with a copy number variation for alpha-synuclein wherein the fibroblast cells are derived from a human with a neurodegenerative disease.

Also described herein is a primary fibroblast cell line derived from a human exhibiting symptoms of Parkinson's disease or Parkinson's-related disease, wherein the primary fibroblast cell line includes alpha-synuclein triplication. Such cell lines are useful as cells with a specific phenotype as in vitro models for neurodegenerative disease.

In various embodiments, the neurodegenerative disease described herein includes Parkinson's disease (PD) or Parkinson's-related disease. The compositions and methods herein have a broad spectrum of utility in clinical applications including, for example, diagnosis of PD, prognosis of PD, determination of treatment efficacy for PD, and selection of a treatment regimen for a subject suffering from PD.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates mitochondrial function in control and mutant fibroblasts. (A) Growth rate of SNCA-Tri was decreased significantly by 24% compared to the controls, *p<0.001 (B) A reduction of mitochondrial Complex I activity in SNCA-Tri culture was approximately 49%, *p<0.0084. (C) Complex I-linked ATP production in patient cells was decreased by 39%, *p<0.0493. (D) There is no significant difference in the mitochondrial content in patient and control fibroblasts measured by quantitative RT-PCR of mitochondrial DNA. Data are presented as mean± standard error of the mean (SEM) compared to the controls (n=9 in the controls; n=9 in SNCA-Tri). p-value of each study was determined by student's unpaired t-test.

FIG. 2 shows SNCA-Tri fibroblasts treated with paraquat (PQ). (A) Under naive conditions, SNCA-Tri fibroblasts showed 33% increase in LDH release (*p<0.0008); after PQ exposure, an increase of LDH release (46%, **p<0.0007) showed reduced cell viability in the patient fibroblasts compared to controls. (B) Mitochondrial membrane potential was reduced by 40% under naive conditions (*p<0.0001) and further impaired by 51% (**p<0.0001) in SNCA-Tri carriers compared to controls after PQ exposure. (C) Cellular ATP levels under naive conditions showed a 47% reduction p<0.0001); after PQ exposure, a 59% decrease of cellular ATP between controls and SNCA-Tri were detected (**p<0.0001). (D) Comparison of % change in untreated and PQ exposed controls or SNCA-Tri. For LDH release, a 27% change in controls was measured, and a 42% change in SNCA-tri, thus a 1.6 fold difference (*p<0.0084); Cellular ATP levels showed a 33% and 56% difference in controls and SNCA-tri, respectively, a 1.7 fold change (**p<0.0032); mitochondrial membrane potential showed a 40% and 61% decrease in controls and SNCA-Tri, respectively leading to a 1.5 fold change (***p<0.0011). (E) After PQ exposure, the level of α-syn mRNA expression increased by 2.6 fold (*p<0.0012) in controls and 7.5 fold (**p<0.0016) in SNCA-Tri, which equaled a 3.2 fold difference (p<0.0039) in SNCA-Tri culture compared to control cultures. Data are presented as mean± standard error of the mean (SEM) compared to the controls (n=9 in the controls; n=9 in the SNCA-Tri). p-value of each study was determined by student's unpaired t-test.

FIG. 3 shows siRNA-mediated knockdown of α-syn in the control and mutant fibroblasts after paraquat (PQ) insult. (A) siRNA knockdown of α-syn assessed by qPCR showed 76% reduction of α-syn mRNA (*p<0.0071). (B) LDH level was decreased by 31% (*p<0.01) with no effect of either scrambled siRNA or Lipofectamine 2000™ (lipofectamine) alone. (C and D) Rescue of the mitochondrial function by SNCA1 after PQ exposure was demonstrated by significantly increasing the level of cellular ATP and mitochondrial membrane potential (37%, *p<0.012 and 36%, *p<0.036) respectively. Data are presented as mean± standard error of the mean (SEM) compared to the controls (n=9 in the controls; n=9 in the SNCA-Tri). p-value of each study was assessed by one-way ANOVA along with Newman-Keuls post-hoc analysis.

### DETAILED DESCRIPTION OF THE INVENTION

Parkinson's Disease (PD) is a movement disorder characterized by gradually progressing bradykinesia, resting tremor, and postural instability with an age-related onset (Gelb et al., Arch. Neurol. 56, 33-39 (1999)). In its typical manifestation, it involves primarily the degeneration and loss of dopaminergic neurons in the substantia nigra, resulting eventually in severe deficiency of the neurotransmitter dopamine. This type of neurodegeneration involves the formation of intracellular inclusion bodies (Lewy bodies) (Formo, J. Neuropathol. Exp. Neurol. 55, 259-272 (1996)), which contain the protein synuclein as a major constituent (Spillantini et al., Nature 388, 839-840 (1997); Baba et al., Am. J. Pathol. 152, 879-884 (1998)). PD can therefore be classified as a distinct protein aggregation disorder affecting specific subpopulations of neurons.

Besides classical PD, Parkinsonism-related disorders have been defined with similar impairment of movement as in PD, but extended symptomatology involving also memory and cognitive functions. In such cases Lewy body formation has spread to cortical areas as well, providing for considerable diagnostic overlap with Dementia with Lewy bodies (DLB). Because of the pervasive involvement of synuclein in Lewy body formation, these diverse disorders are grouped under the term synucleopathies. In spite of this conspicuous association, however, Lewy bodies may be more of a classification feature, reporting a specific pathobiochemistry, rather than a direct cause of neurodegeneration (Jellinger, Biochem. Biophys, Acta 2008; Parkinnen et al., Acta Neuropathol. 116, 125-128 (2008)). On the other hand, the observed commonalities do suggest that certain forms of Parkinson's Disease with Dementia (PDD) are mechanistically related to classical PD. However, there are also forms of PDD with completely unrelated disease biology involving a different form of neurodegeneration based on the pathobiochemistry of the microtubule-associated protein tau (tauopathy), as most clearly exemplified by Frontotemporal Dementia with Parkinsonism caused by mutations in tau protein on chromosome 17 (FTDP-17) (Hutton et al., Nature 393, 702705 (1998)). Hence, in view of the evolving molecular insights into the basis of these neurological disorders the classical clinical diagnoses will become more advantageously replaced by disease-mechanism based classifications, especially if the therapeutic consequences of diagnosis are increasingly less oriented on symptom relief but rather on causative treatment strategies. PD can present with an unknown etiology (idiopathic or sporadic PD) or from patients with a family history of PD (familial PD).

As described, α-synuclein (α-syn) is a major component of Lewy bodies, which are intraneuronal inclusions representing one of the hallmarks of Parkinson's disease (PD). In the basal ganglia of PD brains, in addition to accumulation of Lewy bodies, α-syn accumulates within mitochondria, which manifests as a decrease in Complex I activity. Although the function of α-syn is still unknown, overexpression of the α-syn gene (SNCA) in familial cases of PD due to SNCA duplications or triplications can lead to some clinical and pathological features of PD. However, the mechanism by which α-syn contributes to clinical and pathological features has not been described in the literature.

The compositions and methods herein have a broad spectrum of utility in clinical applications including, for example, diagnosis of PD, prognosis of PD, determination of treatment efficacy for PD, and selection of a treatment regimen for a subject suffering from PD.

### Methods of Screening

The fibroblast cells as described herein may be used as an in vitro model for neurodegenerative disease such as Parkinson's disease. As described herein, the fibroblast cells represent a phenotypic model for Parkinson's disease, thus allowing for rapid and easy evaluation of putative agents for the treatment of neurodegenerative disease such as Parkinson's disease.

A method is disclosed for measuring efficacy of an agent in the treatment of neurodegenerative disease, the method comprising contacting the agent with fibroblast cells containing a copy number variation for alpha-synuclein; detecting a response in the cells; and comparing the response to control cells. In various embodiments, the neurodegenerative disease is Parkinson's disease or Parkinson's-related disease. For example, in various embodiments, the copy number variation is a deletion, an insertion, a complex multi-state variant, a substitution, a transition, a transversion, or a duplication, of one or more nucleotides in the gene for alpha-synuclein. Preferably, the copy number variation is alpha-synuclein triplication. In various embodiments, the fibroblast cells are human-derived, such as from a human with symptoms of the neurodegenerative disease.

In various embodiments, fibroblast cells are present in cell culture with an amount of cells differing from the fibroblast cells, where in the amount of cells differing from the fibroblast cells is selected from less than about 50%, less than about 40%, less than about 30%, less than about 20%, less than about 10%, less than about 5%, less than about 4%, less than about 3%, less than about 2%, less than about 1%, and less than about 0.1%. In various embodiments, the fibroblast cells are essentially free of cells differing from the fibroblast cells.

In various embodiments, the fibroblast cells are present in cell culture with an amount of induced pluripotent stem cells selected from less than about 50%, less than about 40%, less than about 30%, less than about 20%, less than about 10%, less than about 5%, less than about 4%, less than about 3%, less than about 2%, less than about 1%, and less than about 0.1%. In various embodiments, the fibroblast cells are essentially free of induced pluripotent stem cells.

As described herein, the response may be a correction in alpha-synuclein dysfunction. In various embodiments, the response is a change in cell viability, cellular chemistry, cellular function, mitochondrial function, cell aggregation, cell morphology, cellular protein aggregation, gene expression, cellular secretion, cellular uptake, or combinations thereof. For example, in various embodiments, the response is a partial or complete restoration of cell growth. In various embodiments, the response is a partial or complete restoration of mitochondrial function. In various embodiments, the partial or complete restoration of mitochondrial function is selected from the group consisting of increased ATP production, increased mitochondrial membrane potential, increased Complex I activity, and combinations thereof.

In various embodiments of the screening methods as described herein, control cells are fibroblast cells containing a copy number variation for alpha-synuclein without contact with the agent, or the control cells are fibroblast cells containing normal copy number for alpha-synuclein, or both.

Methods of screening the cell lines or cell populations as described herein for an agent to treat a disease or disorder are provided. The methods comprise contacting an agent to be screened with a cell line or cell population described herein, observing a change or lack of change in the one or more cells, where the change or lack of change is correlated with an ability of the agent to treat the disease or disorder. In other words, the change or lack of change can be indicative of an ability of the agent to treat the disease or disorder. Agents to be screened include potential and known therapeutics. Such therapeutics include, but are not limited to, small molecules; aptamers, antisense molecules; antibodies and fragments thereof; polypeptides; proteins; polynucleotides; organic compounds; cytokines; cells; genetic agents including, for example, shRNA, siRNA, a virus or genetic material in a liposome; an inorganic molecule including salts such as, for example, lithium chloride or carbonate; and the like.

In some embodiments, the methods of screening the cell lines or cell populations for an agent to treat a disease or disorder include comparison of the cell lines or populations with another cell line or population. For example, the cell lines or cell populations described herein may be compared to a normal cell line or population, meaning a cell line derived from a subject with no known symptoms or who has not been diagnosed with the disease or disorder of interest. Alternatively, the cell lines or cell populations described herein may be compared to a cell line or population of idiopathic cells, meaning cell lines or populations derived from subjects who present with symptoms of the disease or disorder of interest, or have been diagnosed with the disease or disorder, but who do not have a known genetic variant, and where the cause of the disease or disorder may even be unknown (sporadic or idiopathic). In other embodiments, the methods of screening the cell lines or cell populations for an agent to treat a disease or disorder involve comparison of the cell lines or cell populations to both a normal cell line or cell population and a cell line isolated from a subjecting presenting with an idiopathic/unknown form of disease or population. In some embodiments, the normal cell line or cell population and the idiopathic cell line or population will have been generated using the same protocol as that used to generate the cell line or population as described herein. Thus, the normal cell line or cell population may serve as a control. As well, any change or lack of change in the control cells, idiopathic cells, and cells as described herein upon contacting with an agent may be compared to one another. Patients or groups of patients with idiopathic disease may thereby be compared to patients with genetic variations as described herein with respect to their responsiveness to an agent, to a class of agent, to an amount of agent, and the like. In this way, idiopathic diseases are classified by their responsiveness to agents, yielding information about the etiology of the idiopathic disease and, alternatively or additionally, agents are identified which are effective across one or more classes of disease. It is envisioned that these methods are additionally used to develop treatment regimens for patients or classes of patients with a disease.

In some embodiments, the cell lines or cell populations are screened by staining for a marker and observing a change. Nonlimiting examples of a change or lack of change include a change or lack of change in cell viability, cellular chemistry, cellular function, mitochondrial function, cell aggregation, cell morphology, cellular protein aggregation, gene expression, cellular secretion, or cellular uptake. Cell stains are known to those of skill in the art. Nonlimiting examples include markers of general cytotoxicity in cell viability assays, markers of apoptosis, markers of oxidative stress, markers of mitochondrial function, and combinations thereof. Alternatively, or additionally, screening may be effected by testing for one or more of ATP production, LDH release, activated caspase levels, expression of the gene of interest. In certain embodiments, the screening is for expression of the gene of interest α-synuclein (see, e.g., Andreotti, P. E. et al. Chemosensitivity testing of human tumors using a microplate adenosine triphosphate luminescence assay: Clinical correlation for cisplatin resistance of ovarian carcinoma. Cancer Res. 55, 5276-82(1995); Beckers. B. et al. Application of intracellular ATP determination in lymphocytes for HLA typing. J. Biolumin. Chemilumin. 1, 47-51(1986); Crouch, S. P. M. et al. The use of ATP bioluminescence as a measure of cell proliferation and cytotoxicity. J. Immunol. Meth. 160, 81-8(1993); O'Brien, J. et al. Investigation of the alamar blue (resazurin) fluorescent dye for the assessment of mammalian cell cytotoxicity. Eur. J. Biochem. 267, 5421-6 (2000); Riss, T. and Moravec, R. A. Use of multiple assay endpoints to investigate effects of incubation time, dose of toxin and plating density in cell-based cytotoxicity assays. Assay Drug Dev. Technol. 2, 51-62(2004)).

A wide variety of assays may be used for the purposes described herein, including immunoassays for protein production, amount, secretion or binding; determination of cell growth, differentiation and functional activity; production of hormones; production of neurotransmitters; production of neurohormones; measurement of reactive oxygen species and/or free radical-mediated damage; and the like (see. e.g., Filipov et al., Toxicology 232(1-2):68-78 (2007); Peng et al., J. Neurosci. 26(45):11644-51 (2006); Yan et al., Analysis of oxidative modification of proteins. Curr Protoc Cell Biol., Chapter 7:Unit 7.9 (2002); Armstrong et al., Measurement of Reactive Oxygen Species in Cells and Mitochondria, Methods in Cell Biology, Vol 80, Chapter 18 (2007)).

Fibroblast cells as described herein may be used to screen for agents that enhance or inhibit apoptosis, or the expression of α-synuclein. Typically the candidate agent will be added to the cells, and the response of the cells monitored through evaluation of cell surface phenotype, functional activity, patterns of gene expression, physiological changes, electrophysiological changes and the like. In some embodiments, screening assays are used to identify agents that have a low toxicity in human cells.

A variety of other reagents may be included in the screening assay. These include reagents like salts, neutral proteins, e.g., albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for binding, delivery or effect. Incubations are performed at any suitable temperature, typically ranging from 4 to 40° C., but may be higher or lower than these temperatures. Incubation periods may be selected for optimum activity, but may also be optimized to facilitate rapid high-throughput screening.

Detection of change or lack of change in the cells may utilize staining of cells, performed in accordance with conventional methods. For example, antibodies of interest are added to a cell sample, and incubated for a period of time sufficient to allow binding to the epitope, for example, at least about 10 minutes. The antibody may be labeled with a label, for example, chosen from radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc. One exemplary use of staining in the present methods is described below in more detail.

Cellular gene expression may be assessed following a candidate treatment or experimental manipulation. The expressed set of genes may be compared with control cells of interest, e.g., cells also derived according to the present methods but which have not been contacted with the agent. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of particular mRNAs in cells contacted with agent is compared with the expression of the mRNAs in a control sample.

Gene expression in a test sample may be performed using serial analysis of gene expression (SAGE) methodology, which involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample may also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. Nos. 5,776,683 and 5,807,680.

In another screening method, the test sample is assayed at the protein level. Methods of analysis may include 2-dimensional gels; mass spectroscopy; analysis of specific cell fraction, e.g., lysosomes; and other proteomics approaches. For example, detection may utilize staining of cells or histological sections (e.g., from a biopsy sample) with labeled antibodies, performed in accordance with conventional methods. Cells can be permeabilized to stain cytoplasmic molecules.

### Agents

An agent is described herein for treatment of a neurodegenerative disease formulated in a composition comprising the agent and a carrier suitable for treatment of the neurodegenerative disease, wherein the agent has efficacy in the treatment of the neurodegenerative disease, wherein the efficacy is measured according to the methods described herein.

The term “agent” as used herein describes any molecule, e.g., nucleic acid, protein or pharmaceutical, with the capability of affecting a change in a parameter of interest in the cells of the assay. Generally a plurality of assay mixtures are run in parallel with different agent conditions and/or concentrations to obtain a differential response to the various concentrations. Typically, one of these conditions serves as a negative control, i.e., at zero concentration or below the level of detection. Screening may be directed to known pharmacologically active compounds and chemical analogs thereof.

In various embodiments, the agent is a small molecule. Alternatively, the agent is an antibody, a hybrid antibody, or an antibody fragment. In various embodiments, the agent is a siRNA, and antisense RNA, or an aptamer. In various embodiments, the agent is a protein or a peptide.

Candidate agents encompass numerous chemical classes, including organic molecules. Candidate agents may comprise functional groups necessary for structural interaction with proteins, such as hydrogen bonding, and may include at least one amine, carbonyl, hydroxyl or carboxyl group. In certain embodiments, the candidate agents have at least two of the functional chemical groups. The candidate agents may comprise cyclical carbon or heterocyclic structures and/or aromatic or polyaromatic structures substituted with one or more functional groups. Candidate agents may also be found among biomolecules including, but not limited to: peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay includes a binding assay, one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would often be labeled with a molecule that provides for detection, in accordance with known procedures.

### Fibroblast Cell Cultures

Described herein is a culture of fibroblast cells comprising cell culture media and further comprising fibroblast cells with a copy number variation for alpha-synuclein wherein the fibroblast cells are derived from a human with a neurodegenerative disease. In various embodiments, the neurodegenerative disease is Parkinson's disease or Parkinson's-related disease.

In various embodiments, the cell culture is predominantly one type of cell. For example, in various embodiments, the culture contains an amount of cells differing from the fibroblast cells described herein, where the amount is selected from less than about 50%, less than about 40%, less than about 30%, less than about 20%, less than about 10%, less than about 5%, less than about 4%, less than about 3%, less than about 2%, less than about 1%, and less than about 0.1%. In various embodiments, the cell culture is essentially free of cells differing from the fibroblast cells described herein. In various embodiments, the assessment of screening of putative agents is independent of any effect the putative agents may have on cells differing from the fibroblast cells described herein.

In various embodiments, the culture described herein contains an amount of induced pluripotent stem cells selected from less than about 50%, less than about 40%, less than about 30%, less than about 20%, less than about 10%, less than about 5%, less than about 4%, less than about 3%, less than about 2%, less than about 1%, and less than about 0.1%. In various embodiments, the cell culture is essentially free of induced pluripotent stem cells. In various embodiments, the assessment of screeninv, of putative agents is independent of any effect the putative agents may have on induced pluripotent stem cells.

In various embodiments, the fibroblast cells as described herein have a copy number variation that is alpha-synuclein triplication. In various embodiments, the human from which the fibroblast cells are collected exhibited symptoms of the neurodegenerative disease at a time when the cells were collected.

In various embodiments, the cell culture containing the fibroblast cells as described herein further contains an agent for treatment of neurodegenerative disease.

Also described herein is a primary fibroblast cell line derived from a human exhibiting symptoms of Parkinson's disease or Parkinson's-related disease, wherein the primary fibroblast cell line includes alpha-synuclein triplication. Such cell lines are useful as cells with a specific phenotype as in vitro models for neurodegenerative disease.

Conditioned media, i.e., media in which cells of the methods described herein have been grown for a period of time sufficient to allow secretion of soluble factors into the culture, may be isolated at various stages and the components analyzed for the presence of factors secreted by the cells. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions.

### Pre-Clinical and Clinical Methods

In various embodiments, a method of high-throughput drug screening is disclosed comprising performing the methods described herein, wherein the efficacy of more than one agent is measured, such as a library of agents including a library of known compounds. In various embodiments, a highly automated array is configured to test a library of known and/or unknown compounds. For example, the fibroblast cells as described herein may be used to screen drug libraries. In various embodiments, known compounds are screened for efficacy for re-purposing of a compound with known activity in an assay for a disease other than a neurodegenerative disease such as Parkinson's disease. For example, in various embodiments, efficacy of a number of agents is measured, where the number is selected from more than 10, more than 100, more than 1000, and more than 10,000. In various embodiments, efficacy of combinations of agents is measured.

In various embodiments, the methods described herein are performed by automation.

Also disclosed is a method of pre-clinical or clinical development of a therapeutic for neurodegenerative disease comprising measuring efficacy of more than one agent according to the methods described herein, selecting at least one agent based on the results, and administering at least one agent to an animal model of the neurodegenerative disease. In various embodiments, the selected agent is administered to a human exhibiting no disease symptoms in order to evaluate safety, toxicity, and/or a maximum tolerated dose.

### Methods of Treatment

Methods of treating and/or preventing a disorder (e.g., disease) in a subject in need thereof are provided herein. The methods involve administering to the subject an agent, e.g., identified by the screening methods described herein, in an effective amount to treat or prevent the disorder.

In some embodiments, methods of the disclosure are used to diagnose, theranose, prognose, and/or determine treatment efficacy for a subject. The term “subject” is intended to include organisms, e.g., prokaryotes and eukaryotes, which are capable of suffering from or afflicted with a disease, disorder or condition associated with the activity of alpha-synuclein. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from PD, similar forms of Parkinsonism, and synucleopathies involving Lewy body neurodegeneration. In another embodiment, the subject is a cell.

The term “treat,” “treated,” “treating” or “treatment” includes the diminishment, amelioration, or alleviation of at least one symptom associated with or caused by the state, disorder or disease being treated, e.g., PD, similar forms of Parkinsonism, and synucleopathies involving Lewy body neurodegeneration. In certain embodiments, the treatment comprises the induction of PD or a PD-associated disorder, followed by the activation of the compound of the invention, which would in turn diminish or alleviate at least one symptom associated or caused by the PD or a PD-associated disorder being treated. Treatment can be diminishment of one or several symptoms of a disorder or complete eradication of a disorder.

Nonlimiting examples of additional neurodegenerative disease that may be diagnosed or prognosed by the disclosed methods include Alexander disease, Alper's disease, Alzheimer's disease, Ataxia telangiectasia, Batten disease (also known as Spielmeyer-Vogt-Sjogren-Batten disease), Binswanger's disease, Bovine spongiform encephalopathy (BSE), Canavan disease, Cockayne syndrome, Corticobasal degeneration, Creutzfeldt-Jakob disease, Huntington disease, HIV- or AIDS-associated dementia, Kennedy's disease, Krabbe disease, Lewy body dementia, Machado-Joseph disease (Spinocerebellar ataxia type 3), Multiple sclerosis, Multiple System Atrophy, Myasthenia gravis, sporadic Parkinson's disease, autosomal recessive early-onset Parkinson's disease, Pelizaeus-Merzbacher Disease, Pick's disease, Primary lateral sclerosis, Refsum's disease, Sandhoff disease, Schilder's disease, Schizophrenia, Spielmeyer-VogtSjogren-Batten disease (also known as Batten disease), Spinocerebellar ataxia (multiple types with varying characteristics), Spinal muscular atrophy, Steele-RichardsonOlszewski disease, Stroke, Tabes dorsalis, Angelman syndrome, Autism, Fetal Alcohol syndrome, Fragile X syndrome, Tourette's syndrome, Prader-Willi syndrome, Sex Chromosome Aneuploidy in Males and in Females, William's syndrome, Smith-Magenis syndrome, 22q Deletion, and any combination thereof.

In some embodiments, the disorder is Parkinson's disease (PD) or a PD-related disease. PD-related diseases include diseases, conditions, symptoms or susceptibilities to diseases, conditions or symptoms, that involve, directly or indirectly, neurodegeneration including but not limited to the following: Alpers' disease, Batten disease, Cockayne syndrome, corticobasal ganglionic degeneration, Huntington's disease, Lewy body disease, Pick's disease, motor neuron disease, multiple system atrophy, olivopontocerebellar atrophy, postpoliomyelitis syndrome, prion diseases, progressive supranuclear palsy, Rett syndrome, Shy-Drager syndrome and tuberous sclerosis. Certain PD-related diseases are neurodegenerative diseases that affect neurons in the brain. A PD-related disease may be e.g. a condition that is a risk factor for developing PD, or may be a condition for which PD is a risk factor, or both.

In various embodiments, a method is disclosed for treating neurodegenerative disease in a human comprising administering an agent with efficacy for treatment of the neurodegenerative disease, wherein the efficacy is measured by the methods described herein. In various embodiments, the efficacy is measured prior to administration of the agent to a human for treatment of the neurodegenerative disease.

The agents described herein can be administered in a variety of different ways. The therapeutic agents, identified by the screening methods described herein, may be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds may be achieved in various ways, including intracranial, oral, buccal, rectal, parenteral, intraperitoneal, intravenous, intramuscular, topical, subcutaneous, subdermal, intradermal, transdermal, intrathecal, nasal, intracheal, etc., administration. The active agent may be systemic after administration or may be localized by the use of regional administration, intramural administration, or use of an implant that acts to retain the active dose at the site of implantation. For example, the agent may be intracranially administered using, e.g., an osmotic pump and microcatheter or other neurosurgical device to deliver therapeutic agents to selected regions of the brain under singular, repeated or chronic delivery regimens. In some embodiments, an agent can cross and or even readily pass through the blood-brain barrier, which permits, e.g., oral, parenteral or intravenous administration. Alternatively, the agent can be modified or otherwise altered so that it can cross or be transported across the blood brain barrier. Many strategies known in the art are available for molecules crossing the blood-brain barrier, including but not limited to, increasing the hydrophobic nature of a molecule; introducing the molecule as a conjugate to a carrier, such as transferring, targeted to a receptor in the blood-brain barrier, or to docosahexaenoic acid etc. In another embodiment, an agent is administered via the standard procedure of drilling a small hole in the skull to administer the agent. In another embodiment, the molecule can be administered intracranially or, for example, intraventricularly. In another embodiment, osmotic disruption of the blood-brain barrier can be used to effect delivery of agent to the brain (Nilaver et al., Proc. Natl. Acad. Sci. USA 92:9829-9833 (1995)). In yet another embodiment, an agent can be administered in a liposome targeted to the blood-brain barrier. Administration of pharmaceutical agents in liposomes is known (see Langer, Science 249:1527-1533 (1990); Treat et al., in Liposomes in the Therapy of infectious Disease and Cancer, Lopez-Berestein and Fidler (eds.), Liss, New York, pp. 317-327 and 353-365 (1989). All of such methods are envisioned herein.

Therapeutic agents may include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, nonimmunogenic stabilizers, excipients and the like. The compositions may also include additional substances to approximate physiological conditions, such as pH adjusting and buffering agents, toxicity adjusting agents, wetting agents, and detergents.

Further guidance regarding formulations that are suitable for various types of administration may be found in Remington's Pharmaceutical Sciences, Mace Publishing Company, Philadelphia, Pa., 17th ed. (1985). For a brief review of methods for drug delivery, see, Langer, Science 249:1527-1533 (1990).

The agents identified by the subject methods can be administered for prophylactic and/or therapeutic treatments. Toxicity and therapeutic efficacy of the active ingredient may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are used in some embodiments.

The data obtained from cell culture and/or animal studies may be used in formulating a range of dosages for humans. The dosage of the active ingredient typically lines within a range of circulating concentrations that include the ED50 with low toxicity. The dosage may vary within this range depending upon the dosage form employed and the route of administration utilized.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The effective amount of a therapeutic agent to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD50 animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g., some agents may be taken for extended periods of time on a daily or semi-daily basis, while more selective agents may be administered for more defined time courses, e.g., one, two three or more days, one or more weeks, one or more months, etc., taken daily, semi-daily, semi-weekly, weekly, etc.

A pharmaceutically or therapeutically effective amount of the agent is delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by experimentation according to the art. For purposes of the present method, generally a therapeutic amount may be in the range of about 0.001 mg/kg to about 100 mg/kg body weight, in at least one dose. The subject may be administered in as many doses as is required to reduce and/or alleviate the signs, symptoms, or causes of the disorder in question, or bring about any other desired alteration of a biological system.

### EXAMPLES

As described herein, the effects of α-synuclein (α-syn) gene multiplication on mitochondrial function in human tissue are described. Accordingly, human fibroblasts were taken from a patient with Parkinsonism carrying a triplication in the α-syn gene. Unexpectedly, the cells showed a significant decrease in cell growth compared to matched healthy controls. With regard to mitochondrial function, α-syn triplication fibroblasts exhibited a 39% decrease in ATP production, a 40% reduction in mitochondrial membrane potential, and a 49% reduction in complex I activity. Furthermore, they proved to be more sensitive to the effects of the nigrostrial toxicant paraquat compared to controls. Finally, siRNA knockdown of α-syn resulted in a partial rescue of mitochondrial impairment and reduction of paraquat-induced cell toxicity, demonstrating that α-syn plays a causative role for mitochondrial dysfunction in these patient-derived peripheral skin fibroblasts.

Materials and Methods

Skin Biopsies of Test Subject and Control Subjects

Four mm skin punch biopsies from human test subject with an SNCA triplication (SNCA-Tri) at age 42 and three healthy control individuals (46 yr female sibling of patient, 43 yr male, 35 yr female) were taken using a standard punch biopsy technique from the upper inner arm, an area that is mostly unexposed to direct sunlight. The study and protocol had Institutional Review Board approval and all subjects gave written informed consent for the study. Additional information is provided in Table 1.

TABLE 1
Clinical information of test and control subjects

| Code | Gender | Age | Mutation | Description |
|---|---|---|---|---|
| Con 1 | Female | 35 | None | Healthy control |
| Con 2 | Female | 46 | None | Healthy sibling |
| | | | | control |
| Con 3 | Male | 43 | None | Healthy control |
| SNCA-Tri | Male | 42 | SNCA | Test subject |
| | | | triplication | |

Modified Hoehn and Yahr staging of test subject: The patient had bilateral disease with recovery on pull test, which is equivalent with Hoehn and Yahr Stage 2.5. Unified Parkinson disease rating scale (UPDRS, parts I-III) for test subject at age 42 were 8 points for part 1, non-motor aspects of experiences of daily living, 15 points for part II, motor aspects of experiences of daily living, and 25 points for part III, motor examination. Cognition: Test subject underwent a mini mental state examination (MMSE) at age 42 and scored 30/30 and a Montreal cognitive assessment (MOCA) at age 44 and scored 25/30, which indicated a mild cognitive decline.

Fibroblast Cell Culture

Primary fibroblasts from biopsies were derived from standard skin explant cultures and were banked at low passage numbers. Fibroblasts were cultured in Dulbecco's minimum essential medium, high-glucose (DMEM) with 10% fetal bovine serum, 100 IU/ml penicillin, 100 ug/ml streptomycin, 200 mM glutamine, and 10 mM non-essential amino acids (all purchased from Invitrogen, Carlsbad, Calif.). Experiments were performed at passages 6-12.

Cell Viability and Growth Assay

Cell viability was assessed using the CytoTox-ONE Homogeneous Membrane integrity Assay kit (Cat. No 07890, Promega, Madison, Wis.), which is a fluorescent measure of lactate dehydrogenase (LDH) released from cells with a damaged membrane by conversion of resazurin into resorufin. The assay was performed in a 96-well format according to the manufacturers' instructions. Briefly, 10,000 cells/well of control cells and 12,500 cells/well of SNCA-Tri fibroblasts were used for paraquat (PQ) exposure experiments; 12,500 cells/well were used in siRNA knockout experiments. Cell number was measured using WST-1 proliferation reagent (Roche, Indianapolis, Ind.) in a parallel plate.

The cell growth (5000 cells/well) was measured using WST-1 proliferation reagent. Cells were incubated for 1 hour at 37° C. All measurements were performed on a TECAN GENios (Switzerland). Rate of cell growth was calculated by the ratio of optical density over number of days during exponential growth.

Measurement of Mitochondrial Membrane Potential

Fibroblasts were plated in 96-well plates (6,000 cells/well in the control and 7,500 cells/well in SNCA-Tri cells) and were used for paraquat (PQ) exposure experiments. After 24 hours, cells were changed to galactose medium and cultured for another 24 hours. The mitochondrial membrane potential was measured using the fluorescent dye tetramethylrhodamine methyl ester (TMRM) (Invitrogen) at the final concentration of 150 nM. Cells were incubated for 5 minutes at 37° C. and then washed with phosphate buffered saline (PBS). To remove the plasma membrane contribution from the TMRM fluorescence, each assay was performed in parallel with 10 uM carbonyl cyanide 3-chlorophenylhydrazone (CCCP) (Sigma, St Louis, Mo.), which collapses the mitochondrial membrane potential. All data were expressed as the total TMRM fluorescence minus the CCCP-treated TMRM fluorescence. Cell number was measured using WST-1 proliferation reagent in a parallel plate, according to the manufacturer's instruction. All measurements were performed on a TECAN GENios.

Assessment of Mitochondrial Complex I Activity

Complex I activity of the mitochondrial respiratory chain was assessed by Human Complex I Enzyme Activity Microplate Assay Kit (Cat. No. MS 141, Mitosciences Inc, Eugene, Oreg.) to determine the activity of mitochondrial OXPHOS complex I, according to the manufacturer's instruction. All measurements were performed on the fluorescent plate reader TECAN GENios.

Adenosine Triphosphate Synthesis

Cellular adenosine triphosphate (ATP) levels were measured using the ATPlite kit (Cat. No. 6016943, Perkin Elmer, Waltham, Mass.) based on the reaction of ATP with added lucifcrase and D-luciferin, according to the manufacturer's instruction. ATP synthesis assays were performed on digitonin-treated fibroblasts. Briefly, for paraquat (PQ) exposure, cells were plated (6,000 cells/well in control cells and 7,500 cells/well in SNCA-Tri cells) for 24 hours prior to exposure to PQ.

Toxin Exposure

Cells were exposed to freshly prepared PQ (N,N′-dimethyl-4,4′-bipyridinium dichloride) (Sigma, M2254) at 300 uM final concentration in culture media for 48 hours without media change prior to analysis. According to a dose-response test of control fibroblasts with PQ, 300 uM of PQ showed about 25% cell death at 48 hrs.

Small Interfering RNA-Mediated Knockdown

The sequence of SNCA1 siRNA were used after the method of Fountaine et al, 2008. Custom siRNA (Dharmacon, Lafayette, Colo.) at a concentration of 75 nM (scrambled or α-syn targeted siRNA, SNCA1) was transfected at 90% cell confluency using 0.5 mM Lipofectamine™ 2000 (Invitrogen) according to the manufacturer's instruction. Transfection efficiency was tested to be >90% with siGLO Cyclophilin B control siRNA (Dharmacon) in control fibroblasts.

Quantitative Polymerase Chain Reaction (qPCR)

Total RNA was extracted from fibroblasts using TRIzol® (Invitrogen). cDNA from 0.5 ug total RNA was synthesized by reverse transcriptase (iScript cDNA synthesis kit, BioRad, Hercules, Calif.). Primer sequences of SNCA are SNCA-F: AGTTGTGGCTGCTGCTGAG (SEQ ID NO:1) and SNCA-R: CTCCCTCCTTGGTTTTGGAG (SEQ ID NO:2) and of beta-actin are HuActin-F: CAGCAGATGTGGATCAGCAAG (SEQ ID NO:3) and HuActin-R: GCATTTGCGGTGGACGAT (SEQ ID NO:4). The PCR products of SNCA and beta-actin (as internal control) were amplified using the SYBR Green PCR Master Mix (Applied Biosystems, Carlsbad, Calif.). Amplification of the PCR products was quantitatively measured by the ABI 7000 Sequence Detection System (Applied Biosystems). 2^{-ΔΔCt} method was used in all quantitative PCR analyses.

Statistical Analysis

Data analysis was conducted on three or more biological replicates. Differences among means were analyzed using student's t-test and one-way ANOVA. Newman-Keuls post-hoc analysis was used when differences were observed in ANOVA testing (p<0.05).

### Example 1

### SNCA Triplication Fibroblasts Under Naive Growth Conditions

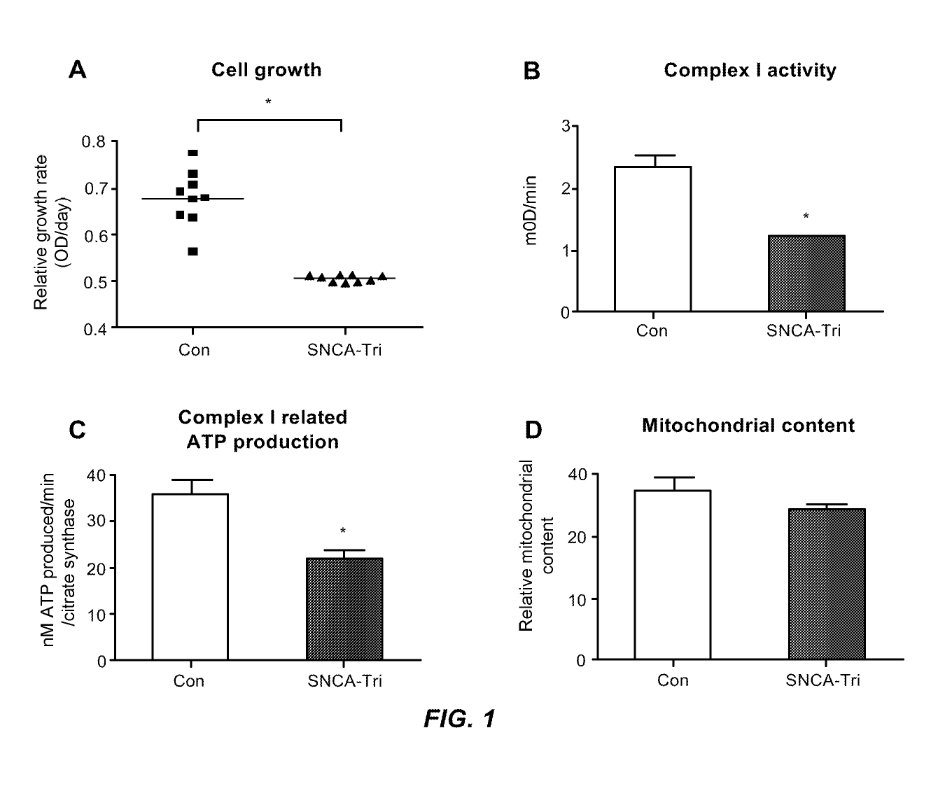

Fibroblast cultures of human test subject with an SNCA triplication (SNCA-Tri) demonstrated a 24% decrease in cell proliferation rate compared to matched healthy control subjects (Con) (FIG. 1A). Owing to the slow growth rate of the SNCA-Tri fibroblasts, whether or not mitochondrial function would be impacted was investigated. Kinetic assessment of the activity of Complex I in cell extracts showed that Complex I activity was 49% lower in the SNCA-Tri cells compared to controls (FIG. 1B).

The Complex I deficiency was associated with impaired mitochondrial ATP production. ATP synthesis was diminished by 39% after specific substrates (pyruvate and malate) for Complex I (FIG. 1C) in the SNCA-Tri fibroblasts. These findings suggest impairment of mitochondrial function and not a decrease in number of mitochondria, since the ratio of mitochondrial DNA/nuclear DNA did not show a significant difference in the control and test subject cultures (FIG. 1D). In summary, the test-subject-derived SNCA triplication fibroblasts demonstrated a pronounced impairment of mitochondrial function under naive culture conditions.

### Example 2

### Increased Susceptibility to Oxidative Stress of SNCA-Tri Fibroblasts after Paraquat Exposure

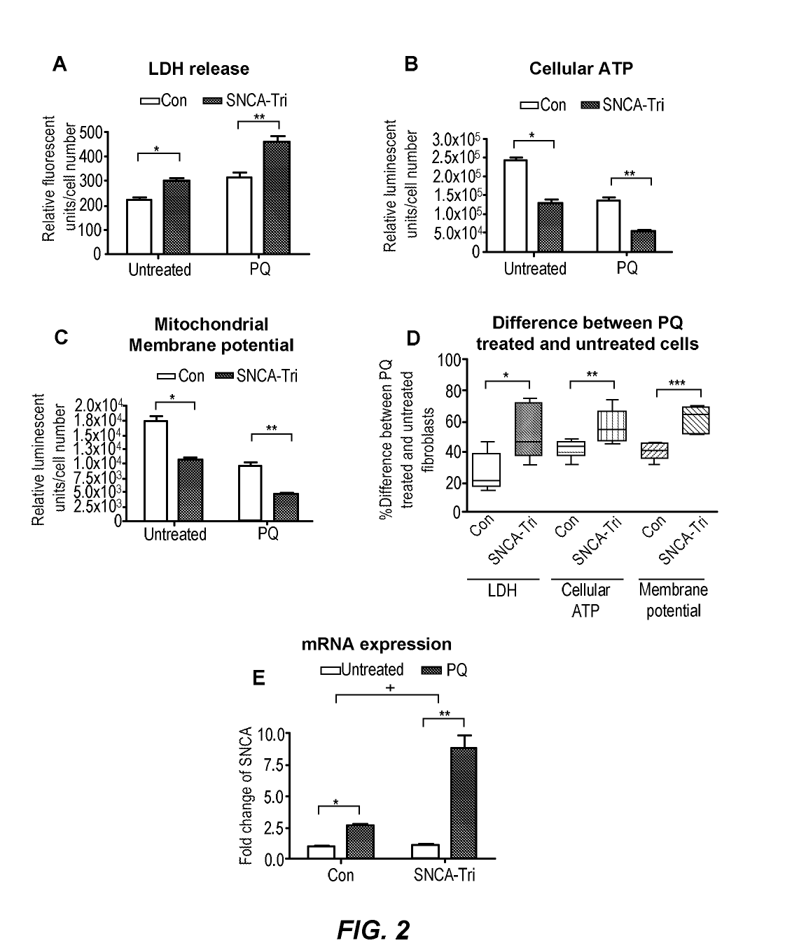

To examine the relationship between mitochondrial dysfunction and oxidative stress, the test-subject SNCA-Tri fibroblasts were tested for susceptibility to oxidative stress compared to control fibroblasts. In these experiments, the cells were exposed to 300 uM of the herbicide paraquat (PQ) for 48 hrs. Cell viability and cell membrane damage were tested using lactate dehydrogenase (LDH) release. Under naive conditions, the SNCA-Tri fibroblasts already showed a slight increase of 33% in LDH release compared to controls 24 hrs after plating. When the cells were treated with PQ, cell viability in SNCA-Tri fibroblasts was greatly affected. Cellular LDH release showed 46% increase after PQ treatment in cells from the SNCA-Tri carrier compared to controls (FIG. 2A). In control fibroblasts compared to SNCA-Tri cells, significant reduction of mitochondrial membrane potential and cellular ATP were observed by 40% and 47% under naive conditions and by 51% and 59% after PQ exposure, respectively (FIGS. 2B and 2C).

The difference (in %) in untreated and PQ exposed fibroblasts was also compared in the control group and the SNCA-Tri fibroblasts (FIG. 2D). The change difference for LDH release in controls was 27% after PQ treatment, whereas the SNCA-Tri fibroblasts showed a 42% increase in LDH release (1.6 fold change). Cellular ATP production was decreased upon PQ treatment in controls by 33% compared to 56% in SNCA-Tri fibroblasts (1.7 fold change). Membrane potential was decreased by 40% in controls upon PQ treatment and 61% in SNCA-Tri fibroblasts (1.5 fold change). These comparisons showed that the SNCA-Tri fibroblasts were more vulnerable to oxidative stress than the matched mutation-negative healthy control cells.

In addition, SNCA transcription levels after PQ exposure were measured. A 2.6-fold increase of α-syn mRNA in the control fibroblasts was observed, which is well described in cell and animal models of Parkinson's disease, but in the SNCA-Tri fibroblasts, SNCA upregulation was unexpectedly high with a 7.5 fold increase in the SNCA-Tri cells compared to naive expression levels, which is 3.2 fold increase of mRNA transcript in SNCA-Tri fibroblasts over control fibroblasts after PQ treatment (FIG. 2E).

Mitochondrial Malfunction can be Partially Reversed by RNAi Knockdown of α-Syn

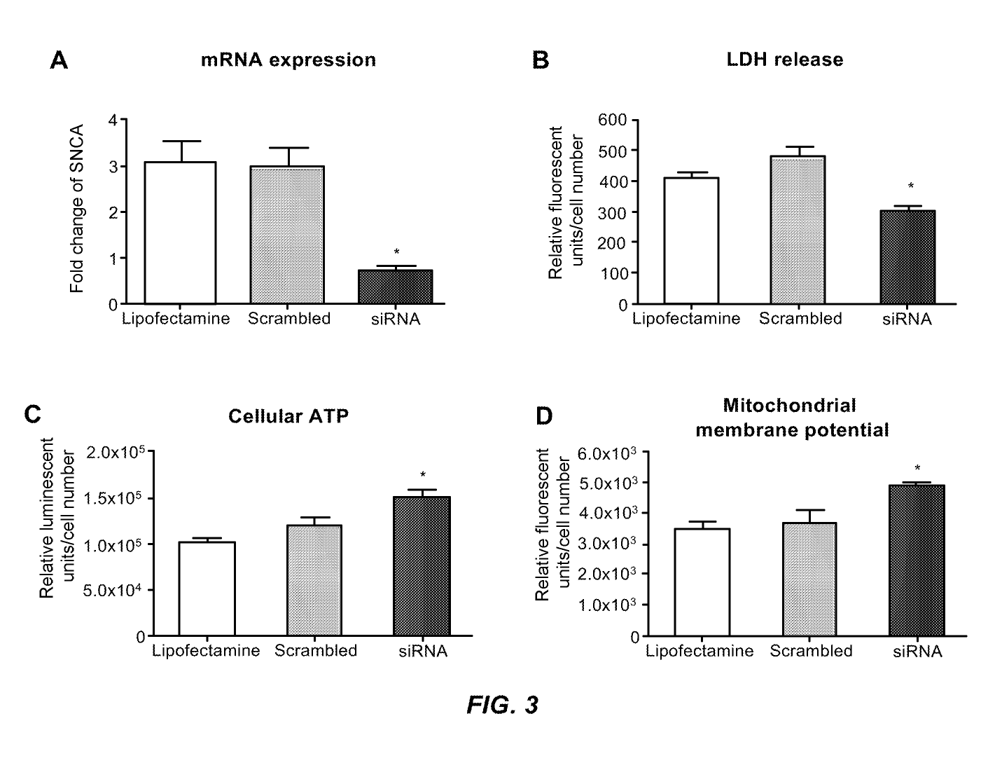

While not wishing to be bound by theory, it was hypothesized that the observed effects are due to the gene multiplication of the SNCA gene. Downregulation of α-syn was tested for amelioration of the observed changes in cell viability, mitochondrial function, and susceptibility to oxidative stress in fibroblasts from the test subject. PQ-exposed SNCA-Tri fibroblasts and controls were transfected with small interfering RNA (siRNA) against SNCA mRNA or scrambled siRNA, and harvested 48 hours after transfection. α-Syn mRNA expression in SNCA-Tri fibroblasts was reduced by approximately 76% 24 hours after siRNA transfection compared to Lipofectamine™ 2000 alone or scrambled siRNA (FIG. 3A). There was no significant difference between mitochondrial function under control conditions and scrambled siRNA or Lipofectamine™ 2000 alone. In the PQ-treated SNCA-Tri culture transfected with siRNA against α-syn after 48 hours, a partial but significant restoration of cell damage was measured by LDH release by 31% (FIG. 3B), the increase in cellular ATP production by 37% (FIG. 3C), and mitochondrial membrane potential by 36% (FIG. 3D). This showed that suppression of α-syn partially reversed mitochondrial malfunction after a neurotoxin insult, thus causatively linking α-syn overexpression due to the SNCA triplication to mitochondrial impairment.

The data provided the first report linking mitochondrial dysfunction and SNCA multiplication in human fibroblasts.

### Example 3

### Phenotype of Fibroblasts with an SNCA Triplication

Without wishing to be hound by theory, mitochondrial impairment may be one of the major disease-associated mechanisms in the etiology of neurodegeneration and Parkinson's disease (PD). Several mitochondrial toxins, such as MPTP or rotenone, inhibit Complex I activity and cause nigrostriatal cell death, which has been utilized in modeling PD in vivo and in vitro. These toxicological models of PD show an increase in α-syn expression and/or an α-syn accumulation.

In humans, a reduction of Complex I activity has been reported in different tissues and brain areas of patients with PD, such as SN and the frontal cortex.

The data herein from peripheral skin fibroblasts from an SNCA triplication carrier support the mechanism of a systemic decrease in mitochondrial function in idiopathic PD, thus demonstrating a cellular phenotype for PD.

In addition, fibroblasts exposed to 300 uM PQ and measured after 48 hours and showed an increase of LDH release indicative of cellular damage. This increase was significantly higher in SNCA-Tri fibroblasts as compared to healthy controls. When assaying mitochondrial function, a significant decrease in membrane potential and ATP levels in SNCA-Tri compared to controls was also observed. While the SNCA triplication itself is already affecting the viability and mitochondrial function of the cells under naive culture conditions, the additional oxidative stress caused by PQ exposure exacerbated the mitochondrial dysfunction in way that was significantly enhanced over that seen in fibroblasts from normal subjects. This provides a phenotypic model of a gene-environment interaction for investigating SNCA gene variants and environmental exposure.

Mitochondrial function and cellular damage was partially rescued after siRNA knockdown of α-syn in fibroblasts after PQ treatment. A significant increase in membrane potential and cellular ATP synthesis was observed as well as a decrease in LDH release supporting the hypothesis that α-syn expression levels were directly related to mitochondrial dysfunction.

Thus, the data showed for the first time a phenotype for mitochondrial impairment in fibroblasts with an SNCA triplication which can be partially rescued by the knockdown of α-syn.

### Example 4

### Assessment of a Putative Therapeutic Agent

Cell Viability and Growth Assay

Cell viability is assessed using the CytoTox-ONE Homogeneous Membrane Integrity Assay kit (Cat. No 67890, Promega, Madison, Wis.), which is a fluorescent measure of lactate dehydrogenase (LDH) released from cells with a damaged membrane by conversion of resazurin into resorufin. The assay is performed in a 96-well format according to the manufacturers' instructions. Briefly, 10,000 cells/well of control cells and 12.500 cells/well of SNCA-Tri fibroblasts are used for exposure experiments with a putative therapeutic agent. Cell number is measured using WST-1 proliferation reagent (Roche, Indianapolis, Ind.) in a parallel plate.

The cell growth (5000 cells/well) is measured using WST-1 proliferation reagent. Cells are incubated for 1 hour at 37° C. All measurements are performed on a TECAN GENios (Switzerland). Rate of cell growth is calculated by the ratio of optical density over number of days during exponential growth.

Measurement of Mitochondrial Membrane Potential

Fibroblasts are plated in 96-well plates (6,000 cells/well in the control and 7.500 cells/well in SNCA-Tri cells) and are used for exposure experiments with a putative therapeutic agent. After 24 hours, cells are changed to galactose medium and cultured for another 24 hours. The mitochondrial membrane potential is measured using the fluorescent dye tetramethylrhodamine methyl ester (TMRM) (Invitrogen) at the final concentration of 150 nM. Cells are incubated for 5 minutes at 37° C. and then washed with phosphate buffered saline (PBS). To remove the plasma membrane contribution from the TMRM fluorescence, each assay is performed in parallel with 10 uM carbonyl cyanide 3-chlorophenylhydrazone (CCCP) (Sigma, St Louis, Mo.), which collapses the mitochondrial membrane potential. All data are expressed as the total TMRM fluorescence minus the CCCP-treated TMRM fluorescence. Cell number is measured using WST-1 proliferation reagent in a parallel plate, according to the manufacturer's instruction. All measurements are performed on a TECAN GENios.

Assessment of Mitochondrial Complex I Activity

Complex I activity of the mitochondrial respiratory chain after exposure to a putative therapeutic agent is assessed by Human Complex I Enzyme Activity Microplate Assay Kit (Cat. No. MS 141, Mitosciences Inc, Eugene, Oreg.) to determine the activity of mitochondrial OXPHOS complex I, according to the manufacturer's instruction. All measurements are performed on the fluorescent plate reader TECAN GENios.

Adenosine Triphosphate Synthesis

Cellular adenosine triphosphate (ATP) levels after exposure to a putative therapeutic agent are measured using the ATPlite kit (Cat. No. 6016943, Perkin Elmer, Waltham, Mass.) based on the reaction of ATP with added lucifcrase and D-luciferin, according to the manufacturer's instruction. ATP synthesis assays are performed on digitonin-treated fibroblasts. Briefly, for exposure to a putative therapeutic agent, cells are plated (6,000 cells/well in control cells and 7,500 cells/well in SNCA-Tri cells) for 24 hours prior to exposure to the putative therapeutic agent.

Toxin Exposure

Cells are exposed to freshly prepared PQ (N,N′-dimethyl-4,4′-bipyridinium dichloride) (Sigma, M2254) at 300 uM final concentration in culture media for 48 hours without media change prior to analysis in combination with a putative therapeutic agent to evaluate the protective efficacy of the putatuive therapeutic agent. To obtain a dose-response test of control fibroblasts, 300 uM of PQ is measured at 48 hrs.

Small Interfering RNA-Mediated Knockdown

The sequence of SNCA1 siRNA is used after the method of Fountaine et al, 2008. Custom siRNA (Dharmacon, Lafayette, Colo.) at a concentration of 75 nM (scrambled or α-syn targeted siRNA, SNCA1) in combination with putative therapeutic agent to evaluate the combined effect. Cells are transfected at 90% cell confluency using 0.5 mM Lipofectamine™ 2000 (Invitrogen) according to the manufacturer's instruction.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of measuring efficacy of an agent in the treatment of neurodegenerative disease, the method comprising:
a. contacting said agent with fibroblast cells containing a copy number variation for alpha-synuclein;
b. detecting a response in the cells; and
c. comparing said response to control cells.

2. The method of claim 1, wherein said neurodegenerative disease is Parkinson's disease or Parkinson's-related disease.

3. The method of claim 1, wherein said copy number variation is a deletion, an insertion, a complex multi-state variant, a substitution, a transition, a transversion, or a duplication, of one or more nucleotides in the gene for alpha-synuclein.

4. The method of claim 1, wherein said copy number variation is alpha-synuclein triplication.

5. The method of claim 1, wherein said fibroblast cells are human-derived.

6. The method of claim 5, wherein said fibroblast cells are derived from a human with symptoms of said neurodegenerative disease.

7. The method of claim 1, wherein said fibroblast cells are present in cell culture with an amount of cells differing from said fibroblast cells, where in said amount of cells differing from said fibroblast cells is selected from less than about 50%, less than about 40%, less than about 30%, less than about 20%, less than about 10%, less than about 5%, less than about 4%, less than about 3%, less than about 2%, less than about 1%, and less than about 0.1%.

8. The method of claim 1, wherein said fibroblast cells are essentially free of cells differing from said fibroblast cells.

9. The method of claim 1, wherein said fibroblast cells are present in cell culture with an amount of induced pluripotent stem cells selected from less than about 50%, less than about 40%, less than about 30%, less than about 20%, less than about 10%, less than about 5%, less than about 4%, less than about 3%, less than about 2%, less than about 1%, and less than about 0.1%.

10. The method of claim 1, wherein said fibroblast cells are essentially free of induced pluripotent stem cells.

11. The method of claim 1, wherein the response is a correction in alpha-synuclein dysfunction.

12. The method of claim 1, wherein the response is a change in cell viability, cellular chemistry, cellular function, mitochondrial function, cell aggregation, cell morphology, cellular protein aggregation, gene expression, cellular secretion, cellular uptake, or combinations thereof.

13. The method of claim 1, wherein said response is a partial or complete restoration of cell growth.

14. The method of claim 1, wherein said response is a partial or complete restoration of mitochondrial function.

15. The method of claim 14, wherein said partial or complete restoration of mitochondrial function is selected from the group consisting of increased ATP production, increased mitochondrial membrane potential, increased Complex I activity, and combinations thereof.

16. The method of claim 1, wherein said control cells are fibroblast cells containing a copy number variation for alpha-synuclein without contact with said agent, or wherein said control cells are fibroblast cells containing normal copy number for alpha-synuclein, or both.

17. The method of claim 1, wherein the agent is selected from a small molecule, a drug, an antibody, a hybrid antibody, an antibody fragment, a siRNA, an antisense RNA, an aptamer, a protein, or a peptide.

18. The method of claim 17, wherein said agent is a small molecule.

19. The method of claim 17, wherein said agent is an antibody, a hybrid antibody, or an antibody fragment.

20. The method of claim 17, wherein said agent is a siRNA, and antisense RNA, or an aptamer.

21. The method of claim 17, wherein said agent is a protein or a peptide.

22. A method of pre-clinical or clinical development of a therapeutic for neurodegenerative disease comprising measuring efficacy of more than one agent according to the method of claim 1, selecting at least one agent based on results of said method of claim 1, and administering said at least one agent to an animal model of said neurodegenerative disease.

23. A method of high-throughput drug screening comprising performing the method of claim 1, wherein efficacy of more than one agent is measured.

24. The method of claim 23, wherein efficacy of a number of agents is measured, wherein said number is selected from more than 10, more than 100, more than 1000, and more than 10,000.

25. The method of claim 23, wherein efficacy of combinations of agents is measured.

26. The method of claim 23, wherein said method is performed by automation.

27. A method of treating neurodegenerative disease in a human comprising administering an agent with efficacy for treatment of said neurodegenerative disease, wherein said efficacy is measured by the method of claim 1.

28. The method of claim 27, wherein said efficacy is measured prior to administration of said agent to a human for treatment of said neurodegenerative disease.

29. An agent for treatment of a neurodegenerative disease formulated in a composition comprising said agent and a carrier suitable for treatment of said neurodegenerative disease, wherein said agent has efficacy in the treatment of said neurodegenerative disease, wherein said efficacy is measured according to the method of claim 1.

30. The agent of claim 29, wherein said agent is a small molecule.

31. The agent of claim 29, wherein said agent is an antibody, a hybrid antibody, or an antibody fragment.

32. The agent of claim 29, wherein said agent is a siRNA, and antisense RNA, or an aptamer.

33. The agent of claim 29, wherein said agent is a protein or a peptide.

34. A culture of fibroblast cells comprising cell culture media and further comprising fibroblast cells with a copy number variation for alpha-synuclein wherein said fibroblast cells are derived from a human with a neurodegenerative disease.

35. The culture of claim 34, wherein said neurodegenerative disease is Parkinson's disease or Parkinson's-related disease.

36. The culture of claim 34, wherein said culture contains an amount of cells differing from said fibroblast cells is selected from less than about 50%, less than about 40%, less than about 30%, less than about 20%, less than about 10%, less than about 5%, less than about 4%, less than about 3%, less than about 2%, less than about 1%, and less than about 0.1%.

37. The culture of claim 34, wherein said culture contains an amount of induced pluripotent stem cells selected from less than about 50%, less than about 40%, less than about 30%, less than about 20%, less than about 10%, less than about 5%, less than about 4%, less than about 3%, less than about 2%, less than about 1%, and less than about 0.1%.

38. The culture of claim 34, wherein said copy number variation is alpha-synuclein triplication.

39. The culture of claim 34, wherein said human exhibited symptoms of said neurodegenerative disease at a time when cells were collected from said human.

40. The culture of claim 34, further comprising an agent for treatment of neurodegenerative disease.

41. A primary fibroblast cell line derived from a human exhibiting symptoms of Parkinson's disease or Parkinson's-related disease, wherein said primary fibroblast cell line includes alpha-synuclein triplication.

42. A method, agent, culture, or cell line according to any of the above claims, wherein said neurodegenerative disease is Parkinson's disease or Parkinson's-related disease.


SEQUENCE LISTING (4 sequences)

SEQ ID NO: 1 - DNA, 19 bp, Artificial Sequence
  Description of Artificial Sequence Synthetic primer
agttgtggctgctgctgag

SEQ ID NO: 2 - DNA, 20 bp, Artificial Sequence
  Description of Artificial Sequence Synthetic primer
ctccctccttggttttggag

SEQ ID NO: 3 - DNA, 21 bp, Artificial Sequence
  Description of Artificial Sequence Synthetic primer
cagcagatgtggatcagcaag

SEQ ID NO: 4 - DNA, 18 bp, Artificial Sequence
  Description of Artificial Sequence Synthetic primer
gcatttgcggtggacgat